(19) United States
(12) Patent Application Publication
(10) Pub. No.: US 2007/0015996 A1
(43) Pub. Date: Jan. 18, 2007

(54) Method for generating and displaying examination images and associated ultrasound catheter

(76) Inventors: Estelle Camus, Erlangen (DE); Martin Kleen, Furth (DE); Thomas Redel, Poxdorf (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/433,710

(22) Filed: May 12, 2006

(30) Foreign Application Priority Data
May 13, 2005 (DE) 10 2005 022 345.1

(51) Int. Cl. A61B 5/05

(52) U.S. Cl. 600/407

(57) ABSTRACT

The invention relates to a method for generating and displaying examination images of a vessel of a patient, comprising the following steps: a) acquiring examination data of the vessel using a first imaging method such as computer tomography, magnetic resonance, or angiography, in particular 3D rotational angiography, b) creating a 3D data set on the basis of the acquired examination data of the first imaging method, c) acquiring examination data and the position of an ultrasound catheter inserted into the vessel, d) creating a 3D data set on the basis of the acquired ultrasound catheter examination and position data as a second imaging method, e) registering the 3D data sets of the first and second imaging method, and f) displaying the registered 3D data sets.

### CROSS REFERENCE TO RELATED APPLICATIONS

This application claims priority of German application No. 102005022345.1 filed May 13, 2005, which is incorporated by reference herein in its entirety.

### FIELD OF THE INVENTION

The invention relates to a method for generating and displaying examination images of a vessel of a patient and an ultrasound catheter suitable for carrying out said method.

### BACKGROUND OF THE INVENTION

Nowadays a whole range of different imaging methods is available, each of which is particularly suitable for a particular examination. For example, 3D rotational angiography is used as an x-ray method for imaging the anatomy of blood vessels and vascular trees. However, this method provides no quantitative information in respect of the blood flow rate in the vessels.

Ultrasound Doppler is used to measure blood flow characteristics such as the blood flow rate, corresponding methods and devices being proposed e.g. in U.S. Pat. No. 5,957,138 and U.S. Pat. No. 5,993,390. The disadvantage of this technique, however, is that the anatomy of the blood vessels under examination is displayed three-dimensionally less precisely and with lower resolution compared to 3D rotational angiography.

Where necessary, therefore, the blood flow rate has hitherto been measured using an ultrasound Doppler method by means of an examination independent of the angiographic examination.

### SUMMARY OF THE INVENTION

The object of the invention is therefore to create a method for generating and displaying examination images of a vessel of a patient whereby both anatomical information and information about the blood flow rate in the vessel can be obtained.

This object is achieved by a method of the type mentioned at the start comprising the following steps:

a) inserting an ultrasound catheter into the vessel to be examined,

b) acquiring examination data of the vessel containing the catheter using an imaging method,

c) creating a 3D data set on the basis of the examination data of the imaging method,

d) acquiring the examination data and position of the ultrasound catheter,

e) creating a 3D data set on the basis of the acquired examination and position data of the ultrasound catheter,

f) registering the 3D data sets of the imaging method and of the ultrasound catheter; and

g) displaying the registered 3D data set.

By means of the method according to the invention, the anatomical information and the information in respect of the blood flow rate is acquired by separate sensors to produce separate 3D data sets which are jointly displayed after registration. This provides a three-dimensional image showing not only the anatomical information but also the blood flow rate as a dynamic process.

The method according to the invention can be used particularly advantageously in the case of an aneurysm in the aorta, for example. For this purpose the ultrasound catheter is inserted into the aorta. The method can also be used for a carotid stenosis, for which purpose the ultrasound catheter is inserted in the jugular vein or an adjacent artery. The method can also be performed for an aneurysm or an AVM (arteriovenous malformation) in the brain, the catheter being inserted into the brain e.g. in an adjacent artery. It is also possible to use the method according to the invention for a stenosis in the coronaries, for which purpose the ultrasound catheter is inserted into the heart in the region of the atrium or ventricle.

To further increase the accuracy of the examination images, with the method according to the invention it can be provided that an electrocardiogram of the patient is recorded. This recording of the cardiac cycle enables the ultrasound examination data to be correlated with the electrocardiogram data. In this way each individual image can be assigned the relevant phase position during the cardiac cycle and, on the basis of this data, ultrasound examination data having the same phase position can be displayed. If the displayed examination images have been recorded during the same phase of the cardiac cycle, the display will not be affected by the different blood flow rates in the course of a cardiac cycle.

According to a further development of the method according to the invention it can be provided that an ultrasound catheter having at least one position sensor is used. The position sensor allows the position of the ultrasound catheter to be determined three-dimensionally so that registration with the three-dimensional data set of the imaging method is facilitated.

For the method according to the invention, at least one ultrasound catheter having a marker can be used, in particular the marker can be implemented as an angiographic marker. Such angiographic markers are visible both on x-ray projections and in the 3D data set of the imaging method. In this way the 3D data set of the ultrasound examination can be registered with the 3D data set of the imaging method so that both 3D data sets are combined in one display.

X-ray methods such as angiography, in particular 3D rotational angiography, are particular suitable as imaging methods. In addition, computer tomography or magnetic resonance can also be used as the imaging method with the method according to the invention.

The invention additionally relates to an ultrasound catheter with at least one ultrasound sensor which is suitable for carrying out the method according to the invention.

According to the invention, the ultrasound catheter has at least one x-ray marker visible during an image recording, in particular an angiography marker, or a magnetic resonance marker and at least one position sensor. The ultrasound catheter according to the invention comprises all the components required on the one hand to acquire the ultrasound data and, on the other, to be able to display the catheter in the image produced by the imaging method.

Particularly advantageously, an x-ray marker or magnetic resonance marker can be spherically shaped. The ultrasound catheter according to the invention preferably comprises a plurality of spherical x-ray markers or magnetic resonance markers distributed around the circumference. According to an alternative embodiment of the invention, a marker can be annularly shaped. The ultrasound catheter according to the invention can preferably have a plurality of annular markers, specifically two.

### BRIEF DESCRIPTION OF THE DRAWINGS

Further advantages and details of the invention will now be explained using exemplary embodiments with reference to the accompanying drawings in which:

FIG. 1 shows a flowchart of the method according to the invention;

FIG. 2 shows a first embodiment of an ultrasound catheter according to the invention; and

FIG. 3 shows a second embodiment of an ultrasound catheter according to the invention.

### DETAILED DESCRIPTION OF THE INVENTION

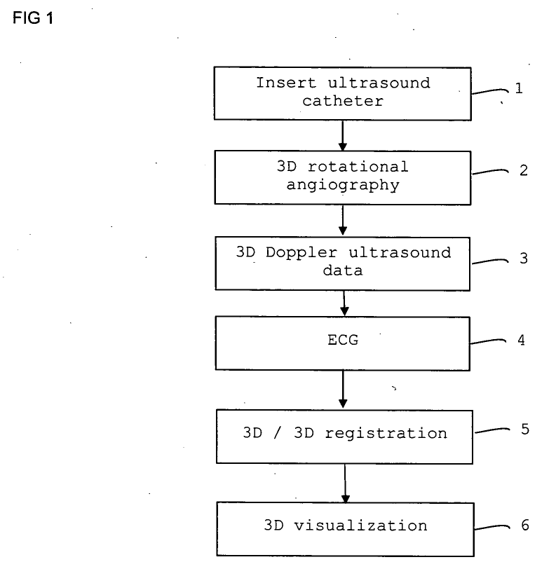

The flowchart illustrated in FIG. 1 shows the essential steps of the method for generating and displaying examination images of a patient's vessel.

After the insertion 1 of the ultrasound catheter in the blood vessel to be examined, x-ray projections are recorded by means of 3D rotational angiography 2 as the imaging method. In the case of an aneurysm in the aorta, the ultrasound catheter is positioned in the aorta. For a carotid stenosis, the ultrasound catheter is used in the jugular vein or in an adjacent artery. In the case of an aneurysm or an AVM (arteriovenous malformation) in the brain, the ultrasound catheter is positioned in the brain or in an adjacent artery.

Doppler ultrasound data 3 is acquired via ultrasound sensors of the ultrasound catheter. Steps 2 and 3, i.e. the performing of 3D rotational angiography and the acquisition of the Doppler ultrasound data 3, take place simultaneously, also consecutively if necessary in the case of other methods. In a first step, the projections are recorded. 3D rotational angiography is then performed, at least the tip of the inserted ultrasound catheter being visible, thereby facilitating the 3D/3D registration performed in step 5. In addition, an electrocardiogram (ECG) 4 is taken in order to monitor to the patient's heart beat and enable the Doppler ultrasound data 3 to be assigned to the relevant phases of the heart beat, thereby allowing for the different blood flow rates as a function of heart beat.

The Doppler ultrasound data 3 acquired in real-time is initially present as two-dimensional data sets. The position data of the ultrasound catheter is simultaneously acquired via a position sensor disposed in the catheter. On the basis of this data and using the electrocardiogram data, a 3D reconstruction of the Doppler ultrasound data 3 is performed.

In step 5, 3D/3D registration of the rotational angiography data set and the Doppler ultrasound data set is performed. By means of registration, the two data sets are matched so that both can be jointly displayed.

This is followed by 3D visualization 6, i.e. a combined 3D displaying of the time resolved Doppler ultrasound data in the rotational angiography data set.

With the method, the x-ray projections, in this case the 3D rotational angiography data, are used to display the anatomy of the vessel under examination. The ultrasound catheter, whose position has been detected by the position sensor, is simultaneously visualized in real-time. A 3D rotational angiography data set is reconstructed from this data.

The ECG data is used to assign the 2D Doppler ultrasound data timewise to a cardiac phase and to construct the associated 3D Doppler ultrasound data set on a time resolved basis, data having the same phase of the heart beat being selected for the visualization.

On the basis of the 2D Doppler ultrasound data acquired in real-time, the blood flow rates in the vessel under examination can be visualized in real-time, and the 3D Doppler ultrasound data set is also reconstructed on the basis of this data.

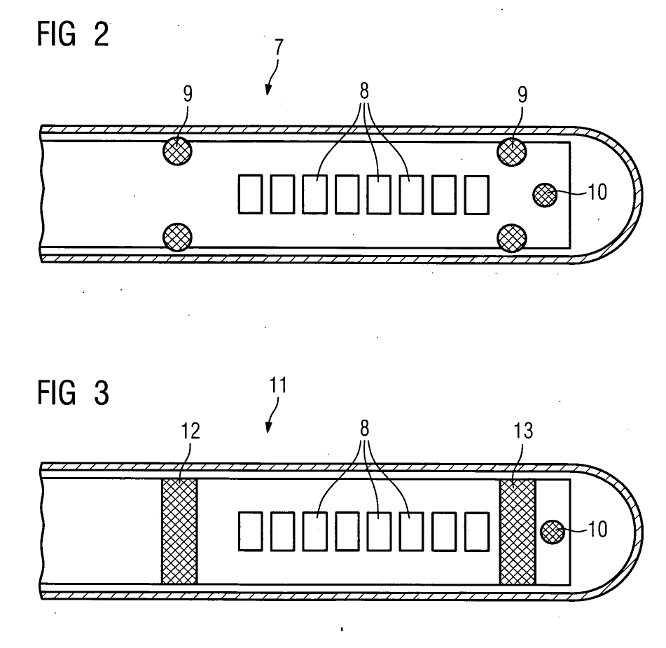

FIG. 2 shows a first embodiment of a catheter.

The catheter 7, of which only the tip is shown in FIG. 2, comprises a plurality of adjacently disposed ultrasound sensors 8 with which the ultrasound signals are detected in the conventional manner. The catheter 7 has four markers 9 which are visible in the angiographic display. These markers 9 are disposed pairwise opposite one another before and after the ultrasound sensors 8. In the area of the tip of the catheter 7 there is disposed a schematically illustrated position sensor 10 which allows three-dimensional detection of the instantaneous position of the catheter 7 in relation to a coordinate system. Position detection takes place in the known manner via magnets (not shown) which are oriented according to the axes of the coordinate system. In other versions of the catheter, a plurality of position sensors may also be present. The position sensor 10 allows three-dimensional reconstruction of the Doppler ultrasound data set, by means of which the two-dimensional Doppler ultrasound data associated with the same heart phase is spatially ordered.

The markers 9 implemented as angiographic markers are visible both on the x-ray projections and in the 3D rotational angiography data set. As these markers 9 are visible in the 3D rotational angiography data set and the position of the markers 9 relative to the tip of the ultrasound catheter 7 is known, the 3D Doppler ultrasound data set can be superimposed on a time resolved basis on the 3D rotational angiography data set.

FIG. 3 shows a second example of an ultrasound catheter. As in the first example, the ultrasound catheter 11 comprises adjacently disposed ultrasound sensors 8 and a position sensor 10. In contrast to the first example, annular markers 12, 13 are provided which are disposed before and after the ultrasound sensors 8. These annular markers 12, 13 are visible both on the x-ray projections and in the 3D rotational angiography data set.

The computational generation of the two 3D data sets is followed by 3D visualization. In the combined 3D display, the 3D rotational angiography data set shows information about the anatomy of the blood vessel and is displayed using transparent colors. The 3D Doppler ultrasound data set shows information about the blood flow rate in the vessel. This information is time resolved, i.e. a particular phase of the cardiac cycle is displayed. The blood flow rate is displayed in color in the 3D rotational angiography data set, e.g. dark red to light red or dark blue to light blue, depending on the blood flow direction.

In other variants of the method, the 3D rotational angiography data set can be replaced by a 3D computer tomography data set or a 3D magnetic resonance data set.

The method and the proposed ultrasound catheter allow x-ray images and Doppler ultrasound data to be combined during one intervention in order to acquire and display information in respect of anatomy and dynamic processes simultaneously. By means of the proposed method, the recording of the two data sets is simplified, as both data sets are obtained simultaneously and discrepancies caused by a different patient position are avoided.

1-12. (canceled)

13. A method for generating and displaying an examination image of a vessel of a patient, comprising:
inserting an ultrasound catheter into the vessel of the patient which is to be examined;
acquiring a first examination data of the vessel of the patient using a first imaging method;
creating a first 3D data set based on the first examination data using the first imaging method;
acquiring an ultrasound examination data of the vessel of the patient using ultrasound as a second imaging method and acquiring a position of the ultrasound catheter;
creating a second 3D data set based on the ultrasound examination data and the position of the ultrasound catheter;
registering the first and second 3D data sets; and
displaying the registered 3D data sets.

14. The method as claimed in claim 13, wherein the registration of the first and second 3D data sets is performed by matching the first and second 3D data sets so that the first and second 3D data sets are jointly displayed.

15. The method as claimed in claim 13, wherein the first image method is selected from the group consisting of: computer tomography, magnetic resonance, or angiography.

16. The method as claimed in claim 15, wherein the angiography is a 3D rotational angiography.

17. The method as claimed in claim 13, wherein an electrocardiogram of the patient is taken prior to displaying the registered 3D data sets.

18. The method as claimed in claim 17, wherein the ultrasound examination data is correlated with the electrocardiogram data.

19. The method as claimed in claim 17, wherein the ultrasound examination data is displayed as a same phase position in a cardiac cycle as detected by the electrocardiogram.

20. The method as claimed in claim 13, wherein the ultrasound examination data is obtained by an ultrasound Doppler method.

21. The method as claimed in claim 13, wherein the ultrasound catheter has a position sensor for three-dimensionally determining the position of the ultrasound catheter.

22. The method as claimed in claim 13, wherein the ultrasound catheter has an x-ray marker.

23. The method as claimed in claim 22, wherein the x-ray marker is an angiographic or magnetic resonance marker.

24. The method as claimed in claim 13, wherein the first 3D data set which contains information of a blood vessel anatomy using the first imaging method is displayed transparently.

25. The method as claimed in claim 13, wherein the second 3D data set which contains a blood flow rate in the vessel using the second imaging method is displayed in color.

26. An ultrasound catheter, comprising:
a position sensor attached to the ultrasound catheter for three-dimensionally determining a position of the ultrasound catheter; and
an ultrasound sensor attached to the ultrasound catheter, the ultrasound sensor having an x-ray marker which is visible during an image recording.

27. The ultrasound catheter as claimed in claim 26, wherein the x-ray marker is an angiographic or magnetic resonance marker that is spherically or annularly shaped.

28. The ultrasound catheter as claimed in claim 27, wherein a plurality of spherically shaped x-ray markers are distributed circumferentially on the ultrasound catheter.

29. The ultrasound catheter as claimed in claim 27, wherein a plurality of annularly shaped x-ray markers are distributed on the ultrasound catheter.

30. The ultrasound catheter as claimed in claim 29, wherein two annularly shaped x-ray markers are distributed on the ultrasound catheter.

31. A device for generating and displaying an examination image of a vessel of a patient, comprising:
an ultrasound catheter inserted into the vessel of the patient which is to be examined;
a first image diagnostic device for generating a first 3D data set of the vessel of the patient;
an ultrasound device as a second image diagnostic device for generating an ultrasound examination data of the vessel of the patient;
a calculator for creating a second 3D data set based on the ultrasound examination data and a position of the ultrasound catheter;
a computing device for superimposing the second 3D data set on the first 3D data set; and
a display device for displaying the superimposed 3D data sets.

32. The device as claimed in claim 31, wherein the first image diagnostic device is selected from the group consisting of: computer tomography, magnetic resonance, or angiography.
